(19) United States
(12) Patent Application Publication
(10) Pub. No.: US 2009/0292356 A1
(43) Pub. Date: Nov. 26, 2009

(54) REDUCED PROFILE INTRAOCULAR LENS

(76) Inventors: Michael Hamlin, Bedford, TX (US); James Scott, Millsap, TX (US); Huong Huynh, Arlington, TX (US)

Correspondence Address: ALCON, IP LEGAL, TB4-8, 6201 SOUTH FREEWAY, FORT WORTH, TX, 76134, US

(21) Appl. No.: 12/470,212

(22) Filed: May 21, 2009

Related U.S. Application Data
(60) Provisional application No. 61/055,356, filed on May 22, 2008.

(51) Int. Cl. A61F 2/16

(52) U.S. Cl. 623/6.46; 623/6.27

(57) ABSTRACT

An improved multifocal design for IOL is provided. A particular embodiment includes an intraocular lens (IOL) with a foldable optic. The IOL also includes multi-hinged haptics coupled to the foldable optic operable to position the IOL within an eye. Each of the multi-hinged haptics includes a first hinge and a second hinge farther from the foldable optic. The first hinge has a first thickness between an anterior side and a posterior side of the multi-hinged haptic, the second hinge has a second thickness between the anterior side and the posterior side of the multi-hinged haptic, and the first thickness is greater than the second thickness. Another embodiment of the present invention provides a method to correct the visual impairment of an aphakic patient by implanting such an IOL.

### RELATED APPLICATIONS

This application claims priority under 35 U.S.C. §119 to provisional application Ser. No. 61/055,356, filed on May 22, 2008, which is incorporated herein by reference.

### TECHNICAL FIELD OF THE INVENTION

The present invention relates generally to the human eye and more particularly to intraocular lenses (IOLs).

### BACKGROUND OF THE INVENTION

The human eye in its simplest terms functions to provide vision by transmitting light through a clear outer portion called the cornea, and focusing the image by way of a lens onto a retina. The quality of the focused image depends on many factors including the size and shape of the eye, and the transparency of the cornea and lens. Age and/or disease often cause the lens to become less transparent. Thus, vision deteriorates because of the diminished light which can be transmitted to the retina. This deficiency in the lens of the eye is medically known as a cataract.

An accepted treatment for this condition is surgical removal of the lens and replacement of the lens function by an Intra-ocular lens (“IOL”). For many years most IOLs were made of polymethylmethacrylate (“PMMA”), a material with good optical characteristics and compatibility with the tissues of the eye. A disadvantage of PMMA, however, is that it is a very rigid material and an incision must be relatively large for implantation of the IOL. If the optical properties of the IOL are not correctly matched to a patient, a second IOL may be required.

All incisions in the eye are accompanied by trauma, and so, although foldable lenses have been a great improvement, there is still a need for an IOL that can be inserted through a smaller incision than previously possible.

### SUMMARY OF THE INVENTION

Embodiments of the present invention provide an improved intraocular lens (IOL). A particular embodiment of the intraocular lens (IOL) includes a foldable optic. The IOL also includes multi-hinged haptics coupled to the foldable optic operable to position the IOL within an eye. Each of the multi-hinged haptics includes a first hinge and a second hinge farther from the foldable optic. The first hinge has a first thickness between an anterior side and a posterior side of the multi-hinged haptic, the second hinge has a second thickness between the anterior side and the posterior side of the multi-hinged haptic, and the first thickness is greater than the second thickness. Another embodiment of the present invention provides a method to correct the visual impairment of an aphakic patient by implanting such an IOL.

In an embodiment having multi hinged haptics, the haptics can include a gusset at the intersection of the haptic and the foldable optic, a distal portion having a widened portion, and a number of hinge-forming elbows spaced intermediate to the gusset in the widened portion. The elbows are sized and shaped to allow the haptic to flex while minimizing buckling and vaulting in the IOL.

In other embodiments, an IOL includes a foldable optic. The IOL also includes multi-hinged haptics coupled to the foldable optic. The hinged optics are angled posteriorly to a plane of optic. The foldable optic vaults posteriorly when the IOL is compressed to a diameter of about 10 mm.

Other advantages of the embodiments of the present invention will become more apparent to one skilled in the art upon reading and understanding the detailed description of the preferred embodiments described herein with reference to the following drawings.

### BRIEF DESCRIPTION OF THE DRAWINGS

For a more complete understanding of the present invention and the advantages thereof, reference is now made to the following description taken in conjunction with the accompanying drawings in which like reference numerals indicate like features and wherein:

FIG. 1 illustrates the anatomy of an eye in which an IOL in accordance with embodiments of the present invention may be implanted;

FIG. 2 depicts an IOL in accordance with embodiments of the present invention;

FIGS. 3A and 3B provide a top-down view and a cross-section of an embodiment of an IOL 300 in accordance with the present invention;

FIGS. 4A and 4B provide a top-down view and a cross-section of an embodiment of an IOL 300 in accordance with the present invention;

FIGS. 5A and 5B provide a top-down view and a cross-section of an embodiment of an IOL 300 in accordance with the present invention;

FIG. 6 provides a logic flow diagram of a method to correct for visual impairments such as aphakia of the eye in accordance with embodiments of the present invention; and

FIGS. 7A and 7B provide a top-down view and a cross-section of an embodiment of an IOL 500 in accordance with the present invent.

### DETAILED DESCRIPTION OF THE INVENTION

Preferred embodiments of the present invention are illustrated in the FIGs., like numerals being used to refer to like and corresponding parts of the various drawings.

An improved design for an IOL is provided. This IOL includes an intraocular lens (IOL) and a number of haptics. The IOL passes optical energy.

The haptics mechanically couple to the IOL in order to position and secure the IOL within the eye.

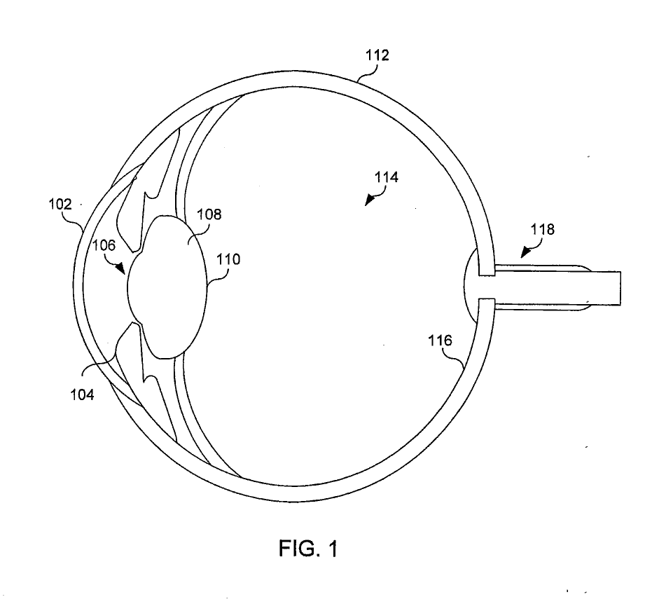

FIG. 1 illustrates the anatomy of an eye into which an improved-design IOL provided by the present invention may be implanted. Eye 100 includes cornea 102, iris 104, pupil 106, lens 108, lens capsule 110, zonules, ciliary body, sclera 112, vitreous gel 114, retina 116, and macula and optic nerve 120. Cornea 102 is a clear, dome-shaped structure on the surface of the eye that acts as a window, letting light into the eye. Iris 104, the colored part of the eye, is a muscle surrounding the pupil that relaxes and contracts to control the amount of light entering the eye. Pupil 106 is the round, central opening of the iris. Lens 108 is the structure inside the eye that primarily acts to focus light on the retina. Lens capsule 110 is an elastic bag that envelops the lens, helping to control lens shape when the eye focuses on objects at different distances. Zonules are slender ligaments that attach the lens capsule to the inside of the eye, holding the lens in place. The ciliary body is the muscular area attached to the lens that contracts and relaxes to control the size of the lens for focusing. Sclera 112 is the tough, outermost layer of the eye that maintains the shape of the eye. Vitreous gel 114 is the large, gel-filled section that is located towards the back of the eyeball, and which helps to maintain the curvature of the eye. Retina 116 is a light-sensitive nerve layer in the back of the eye that receives light and converts it into signals to send to the brain. The macula is the area in the back of the eye that contains functions for seeing fine detail. Optic nerve 118 connects and transmits signals from the eye to the brain.

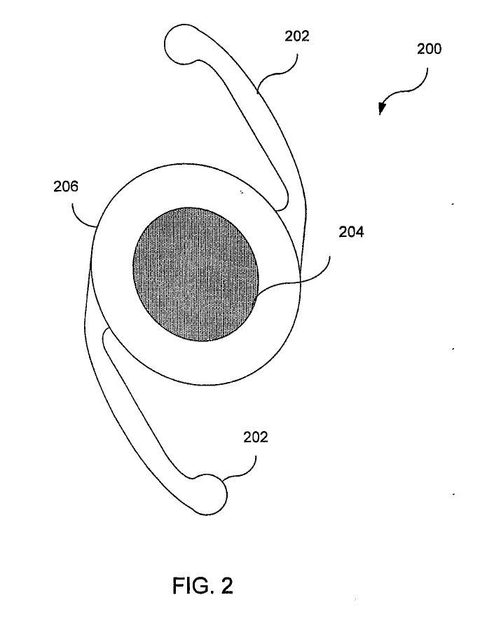

FIG. 2 depicts an IOL 200. IOL 200 is an artificial lens implanted in the eye to restore vision after a natural lens has been removed. The need for the IOL may be due to cataract, disease or accidents. The lens portion (optic) of the IOL may be convex on both sides (biconvex) and made of a soft plastic that can be folded prior to insertion, allowing placement through an incision smaller than the optic diameter of the lens. After surgical insertion into the eye, the lens gently unfolds to restore vision. The supporting arms (haptics) 202 provide for proper positioning of the IOL within the eye.

IOL 200 may be positioned in the posterior chamber of the eye, replacing the natural lens. This position allows IOL 200 to correct the visual impairment of aphakia (absence of the natural lens). IOL 200 may have a biconvex optic that is shaped to provide increased depth of focus. The IOL 200 may be used in adult patients with and without presbyopia, who desire near, intermediate and distance vision with increased independence from glasses following cataract surgery. In particular embodiments, IOL 200 may include a diffractive optic that can deliver quality vision for various lighting situations. In brightly lit conditions, the central portion 204 sends light waves simultaneously to both near and distance focal points, while, in dimly lit conditions, the surrounding area 206 sends greater light energy to distance vision. It should be understood, however, that the haptics according to various embodiments may be used with any kind of optic suitable for a posterior chamber IOL, which include but are not limited to monofocal, multifocal, toric, spheric, aspheric, and accommodative IOLs.

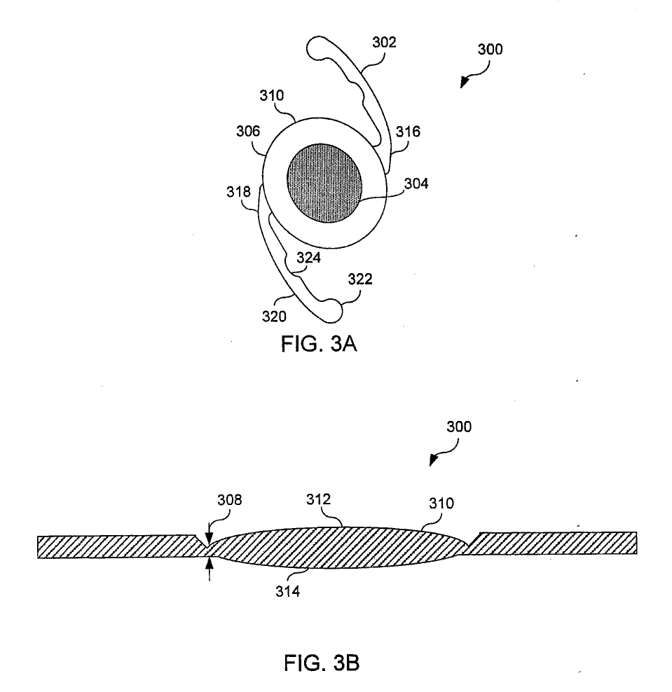

FIGS. 3A and 3B provide a top-down view and a cross-section of an embodiment of an IOL 300 in accordance with the present invention. IOL 300 is operable to be folded and delivered into the capsular bag through a sub 2.1 mm incision and is optically stable after implantation. The optics of the IOL 300 may be convex on both sides (biconvex) and made of a soft plastic that can be folded prior to insertion, allowing placement through an incision smaller than the optic diameter of the lens. After surgical insertion into the eye, the lens gently unfolds to restore vision. The supporting arms (haptics) 302 provide for proper positioning of the IOL within the eye.

Alterations to prior art IOLs that would allow for implantation through a reduced-site incision resulted in IOLs that were non-optically stable after implantation. These prior art attempts merely decreased the thickness of the optic and haptics, creating an unstable optic. Embodiments of the present invention provide unique features that result in an optically stable IOL in the compressed state. These features may be implemented in various combinations and include: (1) a reduced nominal optic edge 308 less than about 0.15 mm thick; and (2) angulated haptic/optic planes, ensuring that any vaulting of optic 306 will occur posteriorly. The novice would expect the lens to vault in the anterior direction because of the angle of the haptics compared to the optic. The design actually creates an unexpected, non-vaulting lens (when compressed to about 10 mm). A further advantageous feature is a multi (e.g., double) hinged haptic design. These features result in an optically sound and stable IOL when compressed to about 10 mm, while maintaining acceptable force (3.0E-04 N) in the haptics. As referred to herein, “about 10 mm” refers to an ordinary range of capsular bag variation in which the IOL would foreseeably be implanted.

IOL 300 may be positioned in the posterior chamber of the eye, replacing the natural lens. IOL 300 may have a biconvex optic In particular embodiments, IOL 200 may include a diffractive optic that can deliver quality vision for various lighting situations. In brightly lit conditions, the central portion 204 sends light waves simultaneously to both near and distance focal points, while, in dimly lit conditions, the surrounding area 206 sends greater light energy to distance vision. It should be understood, however, that the haptics according to various embodiments may be used with any kind of optic suitable for a posterior chamber IOL, which include but are not limited to monofocal, multifocal, toric, spheric, aspheric, and accommodative IOLs.

Haptics 302 may be molded in a single piece from the same material as optics 304 and 306. The material used to make IOL 300 may be any soft biocompatible material capable of being folded. Suitable materials are the hydrogel, silicone or acrylic materials described in U.S. Pat. Nos. 5,411,553 (Gerace, et al.), 5,403,901 (Namdaran, et al.), 5,359,021 (Weinschenk, III, et al.), 5,236,970 (Christ, et al.), 5,141,507 (Parekh) and 4,834,750 (Gupta). Optic 310 has an anterior side 314 and a posterior side 312 and may be of any suitable diameter, with between 4.5 mm and 7.0 mm being preferred and 5.5 mm being most preferred. Optic 310 may also be elliptical or oval. The thickness of optic 310 will vary depending on the dioptic power desired and the index of refraction for the material used, but generally will be between 0.4 mm and 1.5 mm.

IOL 300 attempts to maximize the diameter of optic 310 while minimizing the size of the surgical incision needed to implant IOL 300. The material used to make optic 310 may be modified to absorb ultraviolet radiation, or any other desired radiation wavelength.

Haptics 302 comprise gusset 316, first elbow 318, second elbow 324 and distal portion 320 having widened portion 322. Typical tolerances for these features are around 0.3 mm, so that differences within that tolerance should be considered “about” the nominal value. In one embodiment, the thickness of first elbow 318, second elbow 324 and distal portion 320 of haptic 302 is uniform, and preferably between about 0.30 mm and about 0.60 mm, with between about 0.40 mm and about 0.50 mm being more preferred and about 0.43 being most preferred. Gusset 316, however, has a thickness that is reduced toward anterior side 312 of optic 310. Gusset 316 preferably is between about 0.15 mm and about 0.60 mm thick, with between about 0.25 mm and about 0.35 mm thick being more preferred and about 0.30 mm being most preferred. This reduced thickness generally extends from edge 308 of optic 310. The relatively thin cross section of gusset 316 and edge 308 provides a thinner profile when IOL 300 is inserted through the surgical incision. The reduced thickness of gusset 316 also facilitates fluid circulation (e.g., viscoelastic) between posterior side 314 and anterior side 312 of IOL 300. Alternatively, gusset 316 or optic 310 may be provided with other means (such as holes, grooves, notches, micro-fenestration, or protuberances (all not shown)) to facilitate fluid flow between posterior side 314 and anterior side 312 of IOL 300. The relatively long length and radius of distal portion 320 provides greater contact with the capsular bag for better fixation when IOL 300 is implanted in the eye.

First elbow 318 and second elbow 324 create hinges that allow haptic 302 to flex while minimizing buckling and vaulting of optic 310. The relative thickness of the hinges and other portions of the haptics 302 can be adjusted according to the various considerations disclosed herein as being advantageous for various embodiments of the present invention. In particular embodiments, for example, the second elbow 324 may be made thinner than the first elbow 318, in the manner shown explicitly for the haptic-optic junction, which helps to minimize buckling and to maintain stability and to help the lens to vault only posteriorly. Also, widened portion 322 increases the stiffness of haptic 302 just past elbow 324, thereby increasing the strength of haptic 302 at a critical stress point.

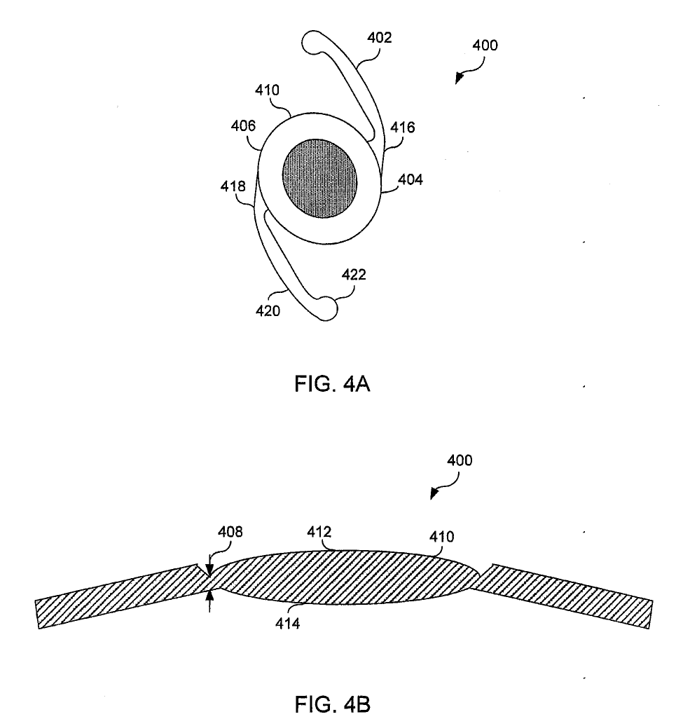

FIGS. 4A and 4B provide a top-down view and a cross-section of an IOL 400 in accordance with embodiments of the present invention similar to that provided in FIGS. 3A and 3B. Haptics 402 contain gusset 416, elbow 418 and distal portion 420 having widened portion 422. In this embodiment, the haptics 402 are angulated relative to the plane of the optic 410. The angulated haptic/optic planes are not parallel. One embodiment angles these planes at about 2.2°. The orientation of these planes ensures that any vaulting of optic 410 will occur posteriorly. Certain embodiments result in a non-vaulting lens (when compressed to about 10 mm).

The relatively long length and radius of distal portion 420 provides greater contact with the capsular bag for better fixation when IOL 400 is implanted in the eye. Elbow 418 creates a hinge allowing haptic 402 to flex while minimizing buckling and vaulting of optic 410. Widened portion 422 increases the stiffness of haptic 402 just past elbow 418, thereby increasing the strength of haptic 402 at a critical stress point.

Advantages of embodiments of the present invention provide for: (1) an IOL that can be folded and delivered into the capsular bag through a sub 2.1 mm incision; (2) a single-piece design that represents a significant reduction in IOL volume without sacrificing mechanical stability; and (3) an IOL that can be fabricated as one piece.

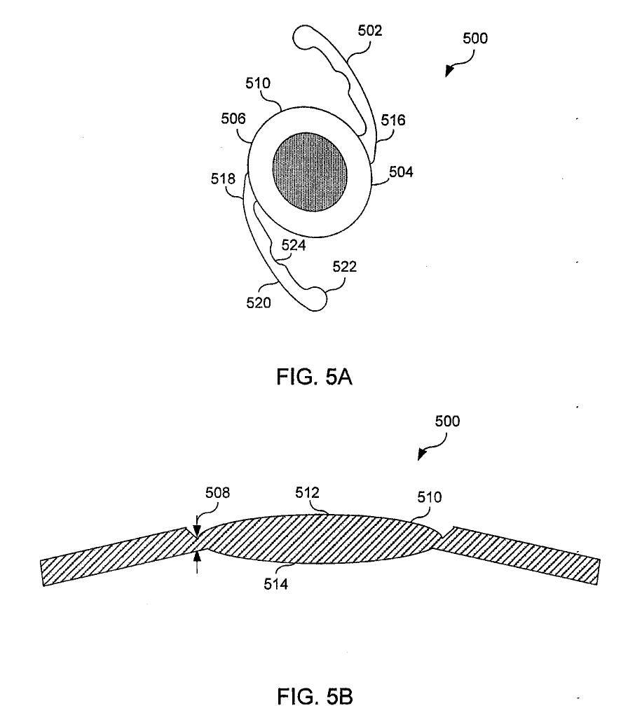

FIGS. 5A and 5B provide a top-down view and a cross-section of an IOL 500 in accordance with embodiments of the present invention incorporating elements provided in FIGS. 3A, 3B, 4A and 4B. Haptics 502 contain gusset 516, first elbow 518, second elbow 524 and distal portion 520 having widened portion 522. In this embodiment, the haptics 502 are multi-hinged and angulated relative to the plane of the optic 510. The angulated haptic/optic planes are not parallel. One embodiment angles these planes at about 2.2°. The orientation of these planes ensures that any vaulting of optic 510 will occur posteriorly. Certain embodiments result in a non-vaulting lens (when compressed to 10 mm).

The relatively long length and radius of distal portion 520 provides greater contact with the capsular bag for better fixation when IOL 500 is implanted in the eye. First elbow 518 and second elbow 524 create hinges allowing haptic 502 to flex while minimizing buckling and vaulting of optic 510. Widened portion 522 increases the stiffness of haptic 502 just past first elbow 518 and second elbow 524, thereby increasing the strength of haptic 502 at a critical stress point.

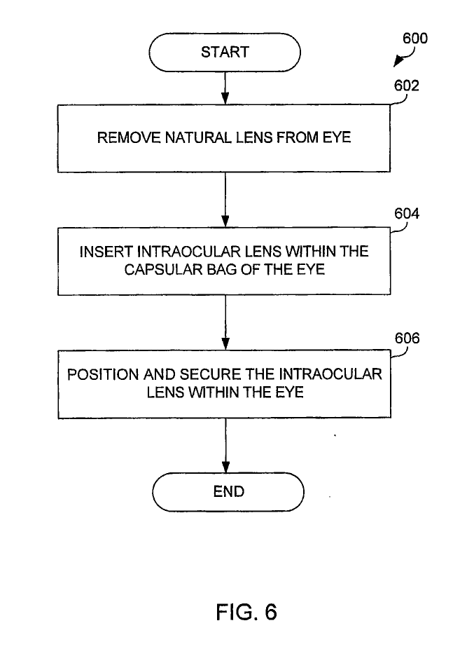

FIG. 6 provides a logic flow diagram of a method to correct for visual impairments such as aphakia of the eye. Operations 600 begin with the removal of a natural lens from an eye in Step 602. The IOL, which may be a multi-focal IOL, may then be inserted within the eye. The lenses of the IOL may be convex on both sides (bi-convex) and made of a soft plastic that can be folded prior to insertion. This folding allows placement through a reduced-size incision wherein the incision is smaller than the optic diameter of the IOL. After surgical insertion into the eye in step 604, the IOL may gently unfold to restore vision. In Step 606, the IOL is positioned and secured within the eye. This may be done with the use of supporting arms (haptics) to provide for proper positioning of the IOL within the eye. Embodiments of the present invention may place or position the IOL in the posterior chamber of the eye to replace the natural lens as shown in FIG. 1. The lens itself may be a multi-focal IOL as discussed previously. This allows patients with and without presbyopia who desire near intermediate and distant vision to experience independence from glasses following surgery such as cataract surgery.

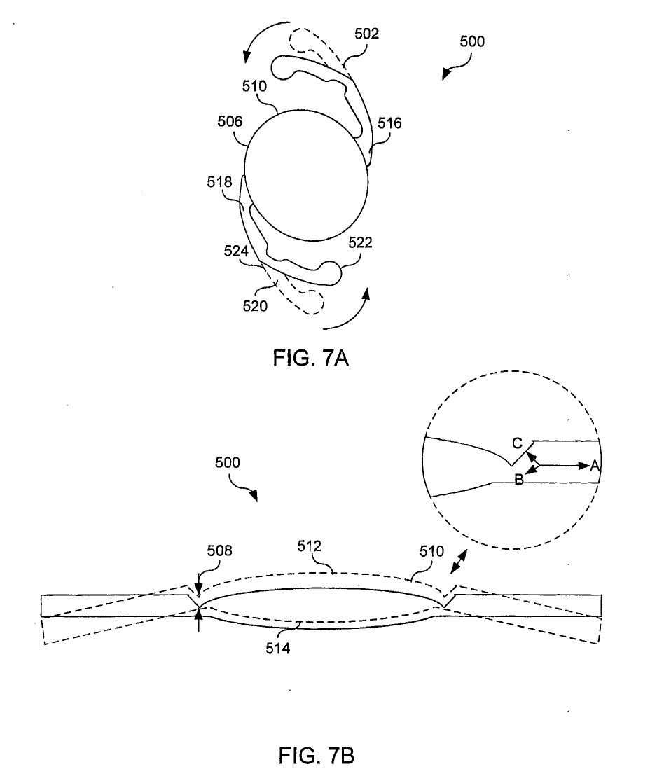

FIGS. 7A and 7B provide a top-down view and a cross-section of an IOL 500 in accordance with embodiments of the present invention as placed within the capsular bag of an eye and incorporating elements provided in FIGS. 3A, 3B, 4A and 4B. FIGS. 7A and 7B show how the forces on IOL 500 equalize and create a stable optical platform during compression. Force A is the residual force. With the force A at an angle not parallel to the optic edge plane, resultant equilibrium forces, labeled B and C, provide the forces that affect the axial displacement, labeled as B, and optic rotation, labeled as C.

Force B, due to the shortened transition area and steeper angle in this region of the haptics counteracts the upward vault expected from the angled haptics. This results in a nearly planar compressed state well below the target of 0.5 mm of vault. The remaining force C is the cause for the rotation of the optic. This unique interaction of the forces due to the new IOL design allows the lens to achieve existing design goals.

In summary, embodiments of the present invention provide an improved design for an IOL. This IOL includes a foldable optic and a number of haptics coupled to the optic. The haptics mechanically couple to the IOL in order to position and secure the IOL within the eye. In one embodiment, the haptics are multi hinged while another embodiment allows the haptics to be placed at an angle to the plane of the optic. The haptics flex while minimizing buckling and vaulting of the IOL in order to position and secure the IOL within the eye.

Although the present invention is described in detail, it should be understood that various changes, substitutions and alterations can be made hereto without departing from the scope of the invention as described by the appended claims.

What is claimed is:

1. An intraocular lens (IOL), comprising:
a foldable optic having an optical axis;
a plurality of multi-hinged haptics coupled to the foldable optic operable to position the foldable optic within an eye, each of the multi-hinged haptics comprising a first hinge and a second hinge farther from the foldable optic, wherein the first hinge has a first thickness between an anterior side and a posterior side of the multi-hinged haptic, the second hinge has a second thickness between the anterior side and the posterior side of the multi-hinged haptic, and the first thickness is greater than the second thickness.

2. The IOL of claim 1, wherein the optic has an edge less than about 0.15 mm.

3. The IOL of claim 1, wherein each of the multi-hinged haptics comprise:
a gusset at the intersection of the haptic and the optic; and
a distal portion having a widened portion; and
wherein the first hinge and the second hinge are spaced intermediate the gusset and the widened portion, and the first hinge and the second hinge each comprise a respective elbow sized and shaped to allow the haptic to flex while minimizing buckling and vaulting of the foldable optic.

4. The IOL of claim 1, wherein the multi-hinged haptics are angled to a plane of the foldable optic.

5. The IOL of claim 1, wherein the multi-hinged haptics are angled at about 2.2° to the plane of the foldable optic.

6. The IOL of claim 1, wherein the IOL is operable to replace a natural lens of the eye.

7. The IOL of claim 1 operable to be implanted within a sub 2.1 mm incision.

8. The IOL of claim 1, wherein the foldable optic vaults posteriorly when the IOL is compressed to a diameter of about 10 mm.

9. The IOL of claim 1, wherein the foldable optic does not vault when the IOL is compressed to a diameter of about 10 mm with a force in the multi-hinged haptics of at least 3.0×10⁻⁴ N.

10. An IOL, comprising:
a foldable optic;
a plurality of multi-hinged haptics coupled to the foldable optic, the multi-hinged haptics angled posteriorly to a plane of optic, wherein the foldable optic vaults posteriorly when the IOL is compressed to a diameter of about 10 mm.

11. The IOL of claim 10, wherein the IOL has an edge less than about 0.15 mm.

12. The IOL of claim 10, wherein the multi-hinged haptics comprise:
a gusset at the intersection of each haptic and the foldable optic;
a distal portion having a widened portion;
a plurality of hinge-forming elbows spaced intermediate the gusset and the widened portion, each elbow sized and shaped to allow the haptic to flex while minimizing buckling and vaulting of the IOL.

13. The IOL of claim 10, wherein the multi-hinged haptics are angled at about 2.2° to the plane of the IOL.

14. The IOL of claim 10, wherein the IOL is operable to replace a natural lens of the eye.

15. The IOL of claim 10 operable to be implanted within a sub 2.1 mm incision.

16. A method to correct visual impairment of aphakia comprising:
removing a natural lens from an eye;
inserting an intraocular lens (IOL) through an incision of a capsular bag of the eye, the IOL comprising a foldable optic and a plurality of multi-hinged haptics coupled to the foldable optic operable to position the IOL within an eye, each of the multi-hinged haptics comprising a first hinge and a second hinge farther from the foldable optic, wherein the first hinge has a first thickness between an anterior side and a posterior side of the multi-hinged haptic, the second hinge has a second thickness between the anterior side and the posterior side of the multi-hinged haptic, and the first thickness is greater than the second thickness; and
positioning and securing the IOL within the eye with the plurality of multi-hinged haptics.

17. The method of claim 16, wherein the IOL has an edge less than about 0.15 mm.

18. The method of claim 16, wherein the IOL is compressed to a diameter of about 10 mm with a force in the multi-hinged haptics of at least 3.0×10⁻⁴ N when inserted.

19. The method of claim 18, wherein the foldable optic does not vault when inserted.

20. The method of claim 18, wherein the foldable optic vaults posteriorly when inserted.

21. The method of claim 16, wherein the multi-hinged haptics are angled at about 2.2° to the plane of the foldable optic.

22. The method of claim 16, wherein the IOL is operable to replace a natural lens of the eye.

23. The method of claim 16 wherein the incision comprises a sub 2.1 mm incision.
